(19) United States
(12) Patent Application Publication
(10) Pub. No.: US 2008/0003269 A1
(43) Pub. Date: Jan. 3, 2008

(54) Poncirus Trifoliata Extract for Preventing and Treating Ischemic Heart Diseases and Pharmaceutical Composition and Health Food Containing the Same

(76) Inventors: Hyun-Su Bae, Seoul (KR); Moon-Kyu Kang, Namyangju-Si (KR); Jong-Hoon Kim, Seoul (KR); Jung-Wan Oh, Seongnam-Si (KR); Chong-Woon Cho, Gyeryong-Si (KR); Chang-Sook Kim, Seoul (KR); Hwa-Jin Lee, Seoul (KR); Hwan-Suck Chung, Seoul (KR); Moo-Chang Hong, Seoul (KR); Min-Kyu Shin, Seoul (KR); Hong-Yeoul Kim, Seoul (KR); Hyun Choi, Seoul (KR); Eun-Jung Ko, Seoul (KR); Chang-Jun An, Seoul (KR)

Correspondence Address: IPLA P.A., 3580 WILSHIRE BLVD., 17TH FLOOR, LOS ANGELES, CA, 90010, US

(21) Appl. No.: 11/570,333

(22) PCT Filed: Jun. 9, 2005
(86) PCT No.: PCT/KR05/01741

§ 371 Date: Dec. 8, 2006

(30) Foreign Application Priority Data
Jun. 9, 2004 (KR) 10-2004-0042192

(51) Int. Cl. A61K 36/752; A61K 9/68; A61P 9/10

(52) U.S. Cl. 424/440; 424/439; 424/736

(57) ABSTRACT

The present invention relates to an Poncirus trifoliata extract having the effect of preventing or treating ischemic heart disease, as well as a pharmaceutical composition and health food for the prevention or treatment of ischemic heart diseases, which contain the Poncirus trifoliata extract. The inventive Poncirus trifoliata extract and the pharmaceutical composition and health food containing the same have the effect of recovering the heart which fails to function properly by ischemic heart diseases. Accordingly, they will be useful for the prevention or treatment of ischemic heart diseases, such as angina and myocardial infarction.

### TECHNICAL FIELD

The present invention relates to an Poncirus trifoliata extract for the prevention or treatment of ischemic heart diseases, and more particularly to, an Poncirus trifoliata extract for recovering the heart failing to function properly by ischemic heart diseases, as well as a pharmaceutical composition and health food containing the same.

### BACKGROUND ART

Heart diseases can be classified into congenital heart diseases and acquired heart diseases. The acquired heart diseases include congestive heart disease (heart failure), ischemic heart diseases (angina, myocardial infraction), valve disease, myocardial disease, endomyocardial disease, arrhythmia, cardiac neurosis, etc.

Among these heart diseases, ischemic heart diseases, which are generally called “coronary heart diseases”, are caused by ateriosclerosis where the arteries become thicker and hardened by plaques resulting from the accumulation of fat and cholesterol on the tunica intima of the coronary arteries supplying blood flow to the heart, and the diameter of the coronary arteries becomes narrower so that a sufficient amount of oxygen cannot be supplied.

If myocardial blood flow abnormalities are caused such that blood flow supply becomes worse and better in a repeated manner, angina will occur. Also, the disruption of plaques leads to acute ischemic syndromes, such as unstable angina, and myocardial infraction where a portion of the coronary arteries is completely clogged so that no blood is supplied to a portion of the heart.

WHO statistics show that 17,000,000 persons every year die due to cardiovascular diseases and are ⅓ of the total number of deaths, which is a numerical value corresponding to the first leading cause of death. In USA, about 1,000,000 persons in 1998 died due to cardiovascular diseases, and thus, corresponded to the first leading cause of death, and particularly, ischemic heart disease accounted for 51% of the cardiovascular diseases (Topal, 1998). In USA, ischemic heart disease is the first leading cause of death in the group of more than 65-year-old persons, and the second leading cause of death in the group of 45-64-year-old persons, and with an increase in the old age population, deaths caused by ischemic heart disease show a tendency to continue to increase. Currently, the ischemic heart disease is a main attack occupying the most majority of death causes in highly advanced countries, and it is reported that the ischemic heart disease is found in 12,000,000 patients only in USA, two of 5 deaths are caused by the ischemic heart diseases, and in Europe and America, 80% of sudden deaths result from the ischemic heart diseases. In Korea, the ischemic heart disease is becoming the greatest cause of death, and mortality resulting therefrom shows a tendency to increase rapidly due to westernized dietary habits, high smoking rate, increased stress and the like.

Agents for treating the ischemic heart disease generally include sympathetic blocking agents, nitrate preparations and calcium antagonists.

Among them, the sympathetic blocking agents (also so-called “beta-blocking agents”) reduce myocardial oxygen demand by reducing heart rate and myocardial contractility by an inhibitory action against sympathetic receptors to blood cathecholamnine, and are most preferably used in acute coronary syndromes and myocardial infarction. This action is known to be particularly effective against an anginal attack occurring in exercise and an anginal attack occurring in sympathetic activation because it prevents increases in blood pressure in exercise and in cardiovascular contractility. However, the combined use of the sympathetic blocking agents with dihydropyridine-based calcium antagonists has the risk of bradycardia and heart block.

Meanwhile, there is no evidence that the nitrate preparations actually reduced mortality or the rate of progression into myocardial infarction, they are first-line drugs which are most widely used together with the sympathetic blocking agents. Their anti-ischemic mechanisms operate through the effects of reducing preload and afterload, expanding blood vessels and collateral vessels, and reducing coronary vasospasms. Also, there is a report that the nitrate preparations inhibit platelet coagulation. Generally, the nitrate preparations are used by intravenous injection while increasing or reducing the dose depending on conditions. However, they can cause a headache and an increase in pulse, and tolerance when injected continuously.

Furthermore, the calcium antagonists (also so-called “calcium blocking agents”) have the effects of coronary vasodilation and blood pressure lowering and can be broadly classified into dihydropyridine (DHP)-based agents and non-dihydropyridine (non-DHP)-based agents. However, the DHP-based preparations, such as nifedipine, cause an increase in myocardial oxygen demand because they have a strong vaso dilation effect but a weak pulse lowering effect or myocardial contractility lowering effect. Actual studies including the use of nifedipine showed that nifedipine caused progression into myocardial infraction and an increase of about 16% in recurrent angina, and thus, the single use of nifedipine has a risk and it must be used in combination with the sympathetic blocking agents. The non-DHP-based agents cause reductions in pulse frequency and myocardial contractility, resulting in a reduction in heart oxygen consumption. These calcium antagonists are used as second-line drugs in patients with contraindications to the sympathetic blocking agents, patients with acute coronary symptoms, who have normal myocardial function, and patients with angina caused by coronary vasospasms.

The above-described drugs used for the treatment of ischemic heart disease have had risks in use because they can cause side effects, such as a sudden reduction in blood pressure, shock death caused by heart function deterioration, general paralysis, and convulsions.

### DISCLOSURE OF INVENTION

Technical Problem

Accordingly, the present inventors have conducted many studies to solve the above-described problems occurring in the prior art, and consequently, found that an Poncirus trifoliata extract, which has been used as a herbal medicinal material, recovers the heart that fails to function properly by ischemic heart diseases, thereby completing the present invention.

It is an object of the present invention to provide an antiischemic Poncirus trifoliata extract which is safe to the human body, has a preventive or therapeutic effect against ischemic heart diseases and has no risk of causing the above-described side effects, as well as a pharmaceutical composition and health food containing the same.

Technical Solution

To achieve the above object, in one aspect, the present invention provides an Poncirus trifoliata extract for the prevention or treatment of ischemic heart disease, which is prepared by extracting Poncirus trifoliata with a solvent.

In another aspect, the present invention provides a pharmaceutical composition and health food for the prevention or treatment of ischemic heart diseases, which contain said extract as an active ingredient.

Advantageous Effects

As described above, the Poncirus trifoliata extract according to the present invention is a natural herbal material harmless to the human body and has the effect of recovering the heart failing to function properly by ischemic heart diseases. Thus, the inventive extract will be useful for the prevention or treatment of ischemic heart disease-related diseases.

### BRIEF DESCRIPTION OF THE DRAWINGS

FIG. 1 is a graphic diagram showing changes in the recovery of blood pressure with time in working heart beat on a test group treated with the inventive Poncirus trifoliata extract and a control group.

FIG. 2 shows a graphic diagram showing changes in the recovery of aortic output with time in working heart beat on a test group treated with the inventive Poncirus trifoliata extract and a control group.

FIG. 3 shows a graphic diagram showing changes in the recovery of coronary perfusion rate with time in working heart beat on a test group treated with the inventive Poncirus trifoliata extract and a control group.

FIG. 4 shows a graphic diagram showing change in the recovery of cardiac output with time in working heart beat on a test group treated with the inventive Poncirus trifoliata extract and a control group.

### BEST MODE FOR CARRYING OUT THE INVENTION

Poncirus trifoliata is the ripened fruit of Poncirus trifoliate, which contains viscous solution, volatile essential oil, etc and is slightly cold in nature and bitter and slightly sour in taste. Poncirus trifoliata has a slower action than the unripened fruit of Poncirus trifoliate, which has green color, and it is used when bowels are irregular due to a phenomenon that “Gi” stays in one place without diffusion, or when a pain is felt due to indigestion resulting from gastritis. Also, it stops a cough, and eliminates dropsy, phlegm, a pain in the side, and clogged vitality in the lower chest. In addition, it is reported to make colonic mobility smooth and to have the effect of treating macules, tears and piles.

On the basis of the above-described pharmacological actions of Poncirus trifoliata, its various applications have been studied. Korean patent laid-open publication No. 2001-37063 discloses an Poncirus trifoliata extract having inhibitory activity against the infection of rotavirus, a main cause for infectious acute diarrhea and enteritis. Also, Korean patent laid-open publication Nos. 2004-10854 and 2004-31491 discloses a concentration for the improvement of constipation and elimination disorders, which is obtained by blending Cascara sagrada powder with Poncirus trifoliata, Angelica sinensis and Achyranthes japonica Nakai, as well as a preparation method and use thereof. However, in these patent publications, there is no mention of use for the prevention and treatment of ischemic heart disease, as described in the present invention.

Hereinafter, the present invention will be described in more detail.

The Poncirus trifoliata extract according to the present invention can be prepared by extraction with water, lower alcohol, hexane, ethyl acetate or a mixture thereof.

More preferably, it can be prepared by an organic solvent extraction process of using an organic solvent to separate volatile or non-volatile substances. The organic solvent extraction process comprises the steps of performing extraction with lower alcohol, hexane, ethyl acetate or a mixture thereof, filtering the extract, concentrating the filtrate, and freeze-drying the concentrate. The lower alcohol is selected from the group consisting of methanol, ethanol and butanol. The extraction processes which can be used in the present invention include percolation extraction, ultrasonic extraction, filtration, reflux extraction, vacuum extraction, and other conventional extraction processes. Moreover, the Poncirus trifoliata extract may also be prepared by a hot water extraction process of using high-temperature hot water to separate soluble substances. The hot-water extraction is preferably carried out at a temperature of 80-100° C. for 1-3 hours, and comprises the steps of adding water to Poncirus trifoliata, filtering and concentrating the solution, and freeze-drying the concentrate. The concentration and freeze-drying steps can be carried out by various conventional methods known in the art.

Mode for the Invention

### EXAMPLE 1

### Preparation of Poncirus trifoliata Extract By Hot-Water Extraction

500 g of a dried powder of Poncirus trifoliata was placed in a flask containing 1 liter of triple-distilled water, and extracted with hot water at 100° C. for 1 hour. The extract was filtered through gauze. The filtrate was concentrated with a vacuum filter (Eyela, Japan) and freeze-dried to prepare the inventive Poncirus trifolita extract. As a result, 110 g of the dried extract was obtained.

### EXAMPLE 2

### Preparation of Poncirus trifoliata Extract By Organic Solvent Extraction

2 liters of an aqueous ethanol solution was added to 1 kg of a dried powder of Poncirus trifoliata and extracted two times with ultrasonic waves for 15 minutes, thus obtaining 3 liters of the extract. The collected extract was filtered through filter paper, and the filtrate was concentrated with a vacuum filter (Eyela, Japan) at 45° C. and 1 atm. The concentrate was freeze-dried to prepare the Poncirus trifoliata extract. As a result, 88 g of the dried extract was obtained.

In order to examine the human body safety of the Poncirus trifoliata extract, an acute toxicity test was first performed.

### TEST EXAMPLE 1

Evaluation of Acute Toxicity of the Poncirus trifoliata Extract to Rats

An acute toxicity test was carried out using 6-week specific pathogen-free (SPF) SD rats. The Poncirus trifoliata extract of the present invention was suspended in a 0.5% methylcellulose solution and orally administered to groups each consisting of five rats in a single dose of 5 g/kg, 10 g/kg and 20 g/kg. After administration of the extract, death, clinicalsymptoms and weight change were observed, and a hema-tological test and hematobiochemical analysis were performed. Upon autopsy, abnormality in abdominal organs and chest organs was visually observed.

As a result, all rats administered with the extract did not show particular clinical symptoms, death, and changes in body weight, as well as toxicity in hematological assay, hematobiochemical analysis and autopsy. As a result, the Poncirus trifoliata extract of the present invention exhibited no toxicity even at a dose of 20 g/kg in all rats, and thus had a 50% lethal dose (LD₅₀) higher than 20 g/kg upon oral administration. This result demonstrates that the Poncirus trifoliata extract is safe.

The inventive Poncirus trifoliata extract was administered orally to white rats according to a conventional method and tested for toxicities in intraabdominal administration and subcutaneous injection. As a result, it was shown that the 50% lethal dose (LD₅₀ ) of the inventive extract was at least 20 g/kg, indicating that the inventive extract is a safe substance.

Then, in order to examine the ability of the Poncirus trifoliata extract to treat ischemic heart disease, changes in blood pressure, aortic output, coronary perfusion rate and cardiac output were measured.

### TEST EXAMPLE 2

Test of the Ability to Treat Ischemic Heart Disease for Myocardial Infarction-Induced Rats

(1) Preparation of Test Group

(i) Isolation of Heart

Sprague-Dawley male rats weighing about 250˜300 gm were purchased and accommodated in a cage at a temperature of 24˜26° C. and a relative humidity of 50˜60% under a 12-hr light/dark cycle controlled with an automatic power unit while the animals were permitted to free access to water and feed. All test procedures were performed according to the ethics of animal experiments. The animals were fasted for two hours before experiments and injected intraabdominally with 5 mg/100 gm bodyweight of pentobarbital to induce anesthesia. The anesthetized rats were fixed to a test bed by tying the legs and arms, and the inguinal region was incised to expose the femoral veins. Then, the exposed femoral veins were injected with 100 units/100 gm bodyweight of heparin. At 60 seconds after the heparin injection, the rats were subjected to median sternotomy to isolate the heart. The isolated heart was immersed in 4° C. physiological saline to induce the arrest of the heart. When the heart was arrested, the airway and gullet around the heart were removed, and then, catheters were inserted into the aorta and the left atrium and fixed with No. 3 silk suture. After ligating the hilum, the lung tissue was separated, and then, the incision marks were made in the pulmonary aorta to prevent a perfusate from filling in the right atrium.

(ii) Ex Vivo Perfusion of Heart

(Preparation and Principal of Ex Vivo Perfusion System)

An ex vivo perfusion circuit of the white rat heart used in this experiment was prepared by attaching a non-working Langendorff persusion system to a working heart perfusion system designed by Neeley and Chain et al.

The non-working ex vivo perfusion system is countercurrent-perfused from an aortic reservoir placed 100 cm above the heart into the heart under a water pressure of 100-cm H₂O, and called the “non-working heart” because it maintains the heart function by coronary perfusion resulting from countercurrent perfusion has no isolation of the heart through the left ventricle. This non-working heart is used for 15 minutes of the initial experimental period and 15 minutes of the first recovery stage after the induction of myocardial infarction, and induces the recovery of the heart from enzyme deficiency in heart isolation and myocardial infarction. The working ex vivo perfusion system refers to a left heart preparation which is perfused from an atrial bubble trap reservoir placed 20 cm above the heart into the left atrium at a water pressure of 20-cm H₂O, and a perfusate flowed in the left atrium flows through the left ventricle into the atrial bubble trap reservoir at a height corresponding to a water pressure of 100-cm H₂O in a flow rate of 20-30 ml/min (this amount is “aortic output”) for each heart beat. In the heart beat, an electrical pacing is not used. This working heart is used for 20 minutes before the induction of myocardial infarction and for 60 minutes after the 15-minute use of the Langendorff persusion system after the induction of myocardial infarction and is critical to compare the heart recovery before and after the induction of myocardial infarction. In this test, aortic perfusate and coronary perfusate are not used in reperfusion.

The heart prepared in the part (i) was connected to the modified Langendorff perfusion system prepared as described above, and subjected to Langendorff perfusion for 15 minutes to remove a blood component from the heart and to balance the concentrations of a solution in the extracellular matrix and a substrate in the perfusate. At this time, infusion into the left atrium was also performed. Furthermore, the controls of heart rate, the maximum aortic systolic pressure and coronary blood flow rate were determined.

Then, the Langendorff perfusion continued to perform while perfusing the left atrium, thus converting the heart into the working heart. After 15 minutes of the working heart before the induction of myocardial infarction, the left artial vessels and the aortic vessels were closed.

(iii) Injection of Poncirus trifoliata extract and induction of myocardial infarction

After the left atrial vessels and the aortic vessels were closed as in the part (ii), 50 ml of the Poncirus trifoliata extract prepared in Example 1 was injected into the coronary artery through an injection cap at a concentration of 1 mg/ml for 3 minutes under a water pressure of 65-cm H₂O and allowed to distribute throughout the heart. Also, to prevent the heart surface from drying, a 37° C. physiological saline was dropped onto the heart surface, and a water jacket of the heart chamber was maintained as a warm jacket using a cardiac local warming method. Also, the temperature of the heart muscle during the entire test process was maintained at 37±1° C., and the left atrial vessels and the aortic vessels were closed and myocardial infarction was induced for 5 minutes.

After the induction of myocardial infarction, the ischemic state of the heart was stopped and the heart was subjected to Langendorff perfusion with a perfusate at 37° C. for 15 minutes. In this case, the coronary perfusate was not subjected to re-perfusion.

After 15 minutes of the non-working perfusion, left atrial perfusion was performed while continuing to perform the Langendorff perfusion, thus the non-working heart to the working heart. The working heart was measured for the recovery of the heart function for 60 minutes. In this case, if the recovery of the heart function was poor due to myocardial injury, the induction of non-working perfusion was not performed so that measurements and observations could be made under the same condition.

(2) Preparation of Control Group

A series of the procedures for preparing the test group were repeated except that myocardial infarction was induced using a perfusate containing no Poncirus trifolita extract.

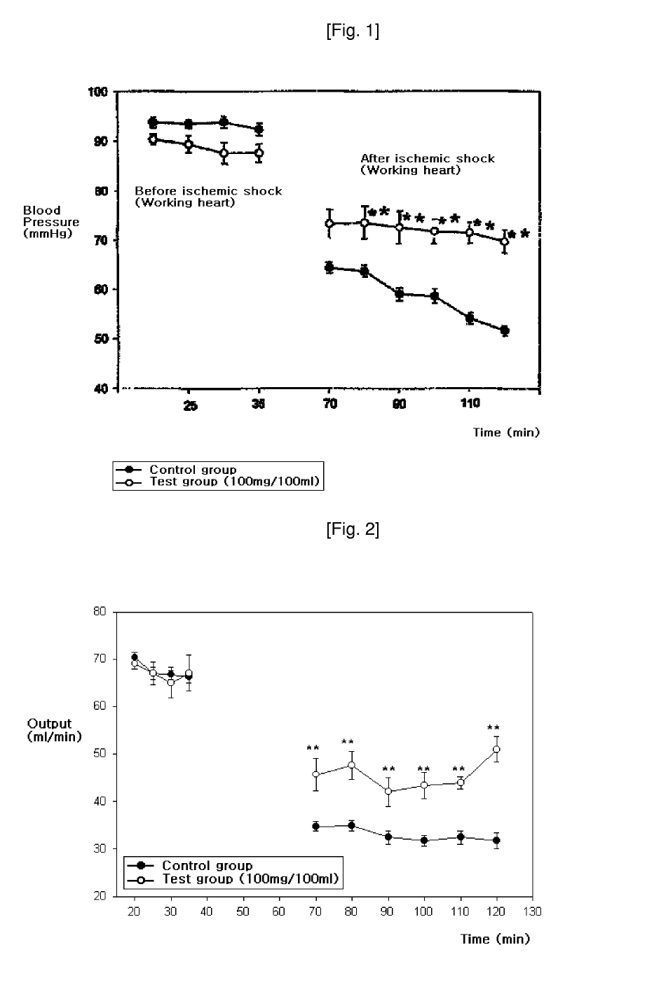

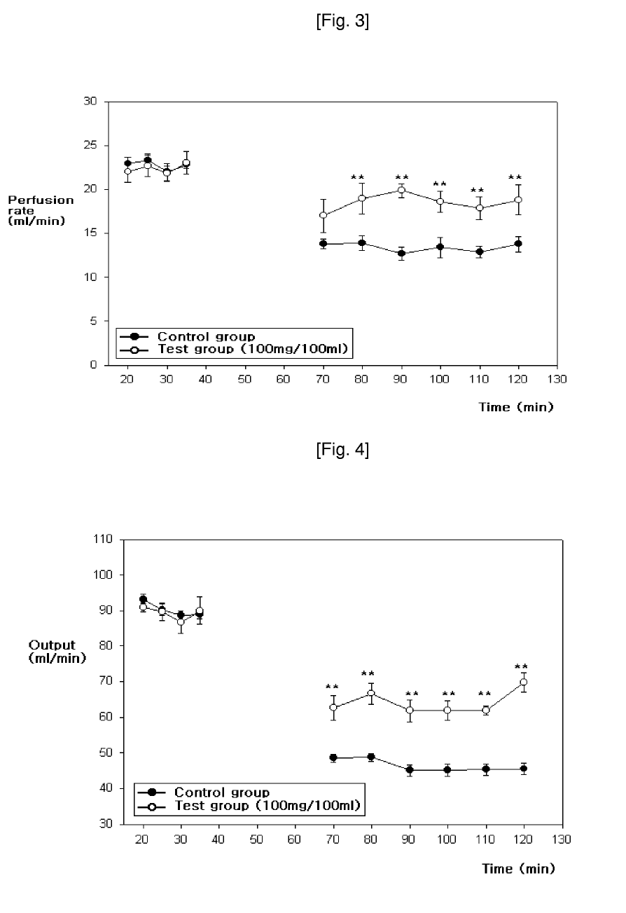

The test group and the control group, each group consisting of 10 members, were measured for each of blood pressure, aortic output, coronary perfusion rate and cardiac output. FIG. 1 shows measurement results for changes in the recovery of blood pressure as a function of time in working heart beat on the test group and the control group. Numerical results shown in Table 1 below indicate mean blood pressure and standard deviations. In FIG. 1, the symbol “**” indicates probability (P)<0.01 as compared to the control group. FIG. 2 shows measurement results for changes in the recovery of cardiac output as a function of time in working heart beat on the test group and the control group, and numerical results shown in Table 1 indicate mean output per minute and standard deviation. FIG. 3 shows measurement results for changes in the recovery of coronary perfusion rate with time in working heart beat on the test group and the control group, and numerical results shown in Table 1 indicate mean perfusion rate per minute and standard deviation. FIG. 4 shows measurement results for changes in the recovery of cardiac output with time in working heart beat on the test group and the control group, and numerical results shown in Table 1 indicate mean output per minute and standard deviation. In FIGS. 2 to 4, the symbol “*” indicates probability (P)<0.05 as compared to the control group, and the symbol “**” indicates probability (P)<0.01 as compared to the control group.

TABLE 1

| | Control group | | | | Test group | | | |
|---|---|---|---|---|---|---|---|---|
| | | | Coronary | | | | Coronary | |
| | Blood | Aortic | Perfusion | Cardiac | Blood | Aortic | Perfusion | Cardiac |
| Time | pressure | output | rate | output | pressure | output | rate | output |
| (min) | (mm Hg) | (ml/min) | (ml/min) | (ml/min) | (mmHg) | (ml/min) | (ml/min) | (ml/min) |
| 20 | 93.70 ± 1.00 | 70.30 ± 1.17 | 22.90 ± 0.82 | 92.20 ± 1.47 | 90.30 ± 1.01 | 69.00 ± 1.15 | 22.05 ± 1.15 | 91.05 ± 1.15 |
| 25 | 93.50 ± 0.87 | 67.00 ± 1.32 | 23.30 ± 0.75 | 90.30 ± 1.51 | 89.40 ± 1.63 | 67.33 ± 2.40 | 22.67 ± 1.20 | 89.67 ± 2.38 |
| 30 | 93.80 ± 1.14 | 66.70 ± 0.94 | 22.00 ± 0.97 | 88.70 ± 1.28 | 87.60 ± 2.09 | 65.00 ± 3.21 | 21.80 ± 0.90 | 86.80 ± 3.19 |
| 35 | 92.30 ± 1.18 | 66.20 ± 1.22 | 22.80 ± 0.39 | 89.00 ± 1.26 | 87.60 ± 1.80 | 67.03 ± 3.79 | 23.06 ± 1.30 | 90.09 ± 3.77 |
| 70 | 64.30 ± 1.11 | 34.70 ± 1.01 | 13.80 ± 0.53 | 48.50 ± 1.11 | 73.30 ± 2.91 | 45.67** ± 3.38 | 17.33 ± 1.86 | 62.67** ± 3.35 |
| 80 | 63.70 ± 1.16 | 34.90 ± 1.15 | 13.90 ± 0.81 | 48.80 ± 1.31 | 73.60** ± 3.32 | 47.67** ± 2.96 | 19.00** ± 1.76 | 66.67** ± 2.93 |
| 90 | 59.00 ± 1.30 | 32.40 ± 1.38 | 12.70 ± 0.72 | 45.10 ± 1.64 | 72.60** ± 3.31 | 42.00** ± 3.06 | 19.87** ± 0.80 | 61.87** ± 3.01 |
| 100 | 58.60 ± 1.46 | 31.70 ± 1.12 | 13.40 ± 1.14 | 45.10 ± 1.68 | 71.80** ± 2.53 | 43.33** ± 2.73 | 18.58** ± 1.20 | 61.91** ± 2.71 |
| 110 | 54.10 ± 1.12 | 32.40 ± 1.40 | 12.90 ± 0.67 | 45.30 ± 1.61 | 71.50** ± 2.13 | 44.02** ± 1.30 | 17.90** ± 1.30 | 61.92** ± 1.30 |
| 120 | 51.60 ± 1.01 | 31.70 ± 1.64 | 13.80 ± 0.88 | 45.50 ± 1.55 | 69.70** ± 2.32 | 51.03** ± 2.65 | 18.80** ± 1.70 | 69.83** ± 2.62 |

\textcircled{1} *Probability (P) < 0.05 compared to the control group
\textcircled{2} **Probability (P) < 0.01 compared to the control group
\textcircled{3} Each numerical value is expressed as mean ± standard deviation

As can be seen from Table 1, the aortic output and cardiac output of the test group treated with the Poncirus trifoliata extract were statistically significantly increased starting from 10 minutes as compared to those of the control group. Also, the blood pressure and coronary perfusion rate of the test group were statistically significantly increased starting from 20 minutes as compared to those of the control group.

Also, before and after the induction of myocardial infarction (ischemic shock), the control group and the test groups were measured for blood pressure, aortic output, coronary perfusion rate and cardiac output, and the measurement values before ischemic shock were expressed in terms of percentages of the measurement values before schemic shock. The results are shown in Table 2 below.

TABLE 2

| | | Control group (%) | | | | Test group (%) | | | |
|---|---|---|---|---|---|---|---|---|---|
| | | | | Coronary | | | | Coronary | |
| | Time | Blood | Aortic | perfusion | Cardiac | Blood | Aortic | perfusion | Cardiac |
| | (min) | pressure | output | rate | output | pressure | output | rate | output |
| Before | ˜20 | 100 | 100 | 100 | 100 | 100 | 100 | 100 | 100 |
| ischemic | | | | | | | | | |
| shock | | | | | | | | | |
| After | 10 | 68.4 | 52.0 | 61.1 | 53.8 | 82.1 | 68.5 | 76.2 | 70.2 |
| ischemic | 20 | 68.1 | 52.3 | 62.5 | 54.1 | 82.4 | 71.5 | 83.6 | 74.7 |
| shock | 30 | 63.1 | 48.6 | 57.1 | 50.0 | 81.3 | 63.0 | 87.4 | 69.3 |
| | 40 | 62.7 | 47.5 | 60.3 | 50.0 | 80.4 | 65.0 | 81.7 | 69.3 |
| | 50 | 58.0 | 48.6 | 58.0 | 50.2 | 80.0 | 66.0 | 78.8 | 69.4 |
| | 60 | 55.2 | 47.5 | 62.1 | 50.5 | 78.1 | 76.5 | 82.7 | 78.2 |

As can be seen from Table 2 above, in the case of the test group treated with the inventive Poncirus trifoliata extract, the blood pressure, aortic output, coronary perfusion rate and cardiac output after ischemic shock almost recovered to the levels before ischemic shock. This suggests that the Poncirus trifoliata extract is effective in treating ischemic heart diseases.

The present pharmaceutical composition for treating ischemic heart diseases includes the Poncirus trifoliata extract which is safe to the human body, and for the prevention or treatment of ischemic heart diseases as an active ingredient. The pharmaceutical composition may be administered orally or parenterally and may be formulated into typical pharmaceutical preparations.

That is, the Poncirus trifoliata extract of the present invention may be formulated into various formulations for oral and parenteral administration upon clinical application. In the formulation, diluents or excipients may be used, which are exemplified by fillers, thickeners, binders, humectants, disintegrators, surfactants, etc.

Examples of solid formulations for oral administration include tablets, pills, powders, granules and capsules. The solid formulations may include, in addition to the Poncirus trifoliata extract, at least one excipient selected from among starch, calcium carbonate, sucrose, lactose, gelatin, etc. Also, the solid formulations may include, in addition toa simple excipient, a lubricant such as magnesium stearate or talc.

Examples of liquid formulations for oral administration include suspensions, internal solutions, emulsions and syrups. The liquid formulations may include, in addition to commonly used simple diluents such as water and liquid paraffin, various excipients which are exemplified by humectants, sweeteners, aromatics and preservatives.

Examples of preparations for parenteral administration include sterile aqueous solutions, non-aqueous solutions, suspensions, emulsions, freeze-dried preparations and suppositories. In the formulation into non-aqueous solutions and suspensions, propylene glycol, polyethylene glycol, vegetable oils such as olive oil, and injectable esters such as ethyl oleate may be used. As a base of suppositories, witepsol, macrogol, Tween 61, cacao fat, lanolin fat, glycerol and gelatin may be used.

The unit dose, may, for example, occurs one, two, three or four times, or a half, third or quarter of an individual dose. The individual dose preferably contains the amount of an effective drugs which is given in one administration and usually corresponds to a whole daily dose or a half, third or quarter of the daily dose.

In the pharmaceutical composition for prevention and treatment of ischemic heart diseases, an effective amount of the Poncirus trifoliata extract ranges from 30 to 700 mg/kg, and preferably 100 to 500 mg/kg, and may be administered once to six times daily. The dosage for a specific patient may vary according to the patient's weight, age, sex, health state and diet, administration duration, administration routes, excretion rates and severity of the illness.

In addition, the present invention provides a health food for treating ischemic heart diseases, comprising the Poncirus trifoliata extract as an active ingredient. In the case of using the present extract as a food, the present extract may be added as it exists or in combination with other food or food ingredients, and may be used suitably according to general methods. Mixed amounts of active ingredients may be suitably determined according to the intended use (preventive, health or therapeutic purposes). Typically, the present extract may be added in an amount of 0.01 to 1 wt %, and preferably 0.1 to 1 wt %, based on the total weight of raw materials used in preparing a food or drink. An effective amount of the present extract may be determined based on an effective amount of the pharmaceutical composition. When consumed for a long period of time for health and sanitary purposes or health control, the present extract may be used in an amount lower than the range. Also, it is apparent that the present extract can be used in an amount higher than the range because the active ingredient carries no safety risk.

The type of the food is not particularly limited. Examples of foods to which the present extract can be added include meats, sausages, breads, chocolates, candies, snacks, confectionary, pizza, instant noodles, other noodles, gums, dairy products including ice creams, various soups, beverages, teas, drinks, alcoholic beverages and vitamin complexes, as well as traditional therapeutic preparations for use as an antianemic, a body function-strengthening agent, a skin whitening agent, and the like.

Also, it can be used in various herbal medicinal formulations, such as Gi-Kag-San, Yeuk-Shin-Hwan, Ji-Cyl-Do-Chae-Tang, and Ji-Cyl-Chong-Baek-Gae-Ji-Tang.

Pharmaceutical formulations and health foods containing the Poncirus trifolita extract were prepared in Formulation Examples 1-7 below, but the scope of the present invention is not limited to these Examples.

### FORMULATION EXAMPLE 1

### Preparation of Soft Capsules

A soft capsules were prepared according to a soft capsule preparation method described in General Rules for Preparation in a guidebook, Korean Pharmacopoeia, using 100.0 mg per capsule of the Poncirus trifolita extract prepared in Example 1, 175.0 mg of soybean oil, 45.0 mg of cera flava, 127.5 mg of hydrogenated palm oil, 21.0 mg of soybean phospholipids, 212.0 mg of gelatin, 50.0 mg of glycerin (gravity: 1.24), 76.0 mg of di-sorbitol, 0.54 mg of methyl-paraoxybenzoate, 0.90 mg of propylparaoxybenzoate, 0.56 mg of methylvanillin, and a proper amount of yellow no. 203 per capsule.

### FORMULATION EXAMPLE 2

### Preparation of Tablets

100.0 mg of the Poncirus trifoliata extract prepared in Example 1, 90.0 mg of corn starch, 175.0 mg of lactose, 15.0 mg of L-hydroxypropylcellulose, 5.0 mg of polyvinylpyrolidone 90 and a proper amount of ethanol were homogeneously mixed, granulated by wet granulation, mixed with 1.8 mg of magnesium stearic acid, and forced into 400 mg tablets.

### FORMULATION EXAMPLE 3

### Preparation of Capsules

100.0 mg of the Poncirus trifoliata extract prepared in Example 1, 83.2 mg of corn starch, 175.0 mg of lactose and 1.8 mg of magnesium stearic acid were homogeneously mixed, and filled into capsule shells at 360 mg per capsule.

### FORMULATION EXAMPLE 4

Preparation of Chewing Gum

Chewing Gum was prepared according to a general method using 0.24˜0.64% of the Poncirus trifoliata extract prepared in Example 1, 20% of gum base, 1% of a fruit aromatic, 2% of water and the balance of sugar.

### FORMULATION EXAMPLE 5

### Preparation of Ice Cream

Ice cream was prepared according to a general method using 0.24˜0.64% of the Poncirus trifoliata extract prepared in Example 1, 10.0% of milk fat, 10.8% of SNF (Solids Not Fat), 12.0% of sugar, 3.0% of starch syrup, 0.5% of an emulsion stabilizer (span), 0.15% of an aromatic (strawberry) and the balance of water.

### FORMULATION EXAMPLE 6

### Preparation of Beverage

A beverage was prepared according to a general method using 0.48˜1.28 mg of the Poncirus trifoliata extract prepared in Example 1, 522 mg of honey, 5 mg of thioctic acid amide, 10 mg of nicotinic acid amide, 3 mg of riboflavin hydrochloride sodium, 2 mg of pyridoxine hydrochloride, 30 mg of inositol, 50 mg of orotic acid and 200 ml of water.

### FORMULATION EXAMPLE 7

### Preparation of Sausage

Sausage was prepared according to a general method using 0.24˜0.64% of the Poncirus trifoliata extract prepared in Example 1, 27.5% of chicken, 3.5% starch, 1.7% of soybean proteins, 1.62% of edible salt, 0.5% of glucose, 0.94˜1.34% of another additive (glycerin) and the balance of pork.

1. An Poncirus trifoliata extract for the prevention or treatment of ischemic heart diseases, which is prepared by extracting Poncirus trifoliata with a solvent.

2. The Poncirus trifoliata extract of claim 1, which is prepared by extracting Poncirus trifoliata with hot water.

3. The Poncirus trifoliata extract of claim 1, wherein the solvent is selected from the group consisting of water, lower alchol, hexane, ethyl acetate and a mixture thereof.

4. A pharmaceutical composition for the prevention or treatment of ischemic heart diseases, comprising the Poncirus trifoliata extract of claim 1 as an active ingredient.

5. A health food for the prevention or treatment of ischemic heart diseases, comprising the Poncirus trifoliata extract of claim 1 as an active ingredient.
